(19) United States
(12) Patent Application Publication
(10) Pub. No.: US 2012/0146009 A1
(43) Pub. Date: Jun. 14, 2012

(54) NOVEL COMPOUND, FIELD-EFFECTIVE TRANSISTOR, SOLAR CELL, METHOD FOR PRODUCING SAID COMPOUND, FIELD-EFFECTIVE TRANSISTOR, AND SOLAR CELL, COMPOSITION FOR ORGANIC SEMICONDUCTOR LAYER, AND COMPOSITION FOR P-TYPE SEMICONDUCTOR LAYER

(75) Inventors: Tetsuji Itoh, Osaka (JP); Shigeru Aomori, Osaka (JP)

(73) Assignee: Sharp Kabushiki Kaisha, Osaka (JP)

(21) Appl. No.: 13/390,925

(22) PCT Filed: Aug. 19, 2010
(86) PCT No.: PCT/JP2010/064023

§ 371 Date: Feb. 16, 2012

(30) Foreign Application Priority Data
Oct. 28, 2009 (JP) 2009-247786

(51) Int. Cl. H01L 51/30; H01B 1/12; H01L 51/48; C07D 241/38; H01L 51/40; H01L 51/46

(52) U.S. Cl. 257/40; 438/99; 136/263; 438/82; 544/342; 252/500; 257/E51.025; 257/E51.026

(57) ABSTRACT

A compound of the present invention is represented by the following formula (1):

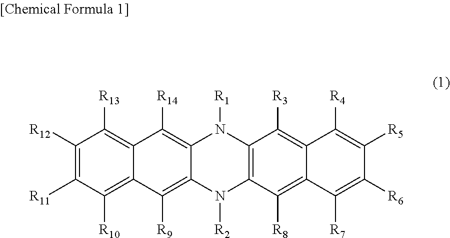

(where: R₁ and R₂ represent, independently, a substitutable C₁ to C₂₀ aliphatic hydrocarbon group; and R₃ through R₁₄ represent, independently, one of a hydrogen atom, a halogen atom, a substitutable aliphatic hydrocarbon group, and a substitutable aromatic hydrocarbon group). It is therefore possible to provide a novel compound which can be used as an organic semiconductor material.

### TECHNICAL FIELD

The present invention relates to a novel organic semiconductor compound and a semiconductor device including the novel organic semiconductor compound.

### BACKGROUND ART

As an organic semiconductor material used as an active layer of a semiconductor device (such as a field effect transistor (FET)), there have been known various compounds having a hole transport property.

For example, Patent Literature 1 describes that a semiconductor layer of an organic semiconductor device is made from pentacene. Further, Patent Literature 2 describes poly(3-octylthiophene) as a polymer organic semiconductor from which a semiconductor layer of a field effect transistor is made. Furthermore, Non-Patent Literature 1 describes that a semiconductor layer of an organic FET device is made from dihydrodiazapentacene (DHDAP). Moreover, Patent Literature 3 describes several condensed polycyclic aromatic heterocyclic compounds which can be used as a hole transport layer of an organic light emitting device.

### CITATION LIST

### Patent Literature

[Patent Literature 1]
Japanese Patent Application Publication, Tokukai, No. 2001-94107 A (Publication Date: Apr. 6, 2001)

[Patent Literature 2]
Japanese Patent Application Publication, Tokukaihei, No. 6-177380 A (Publication Date: Jun. 24, 1994)

[Patent Literature 3]
Specification of US Patent Application No. 2005/0014018A1 (Publication Date: Jan. 20, 2005)

### Non-Patent Literature

[Non-Patent Literature 1]
Chem. Mater. 2009, 21, 1400

### SUMMARY OF INVENTION

### Technical Problem

However, the organic semiconductor materials described in Patent Literatures 1 and 2 are unstable and are likely to be oxidized in an atmosphere. Accordingly, a semiconductor device employing such a material is likely to have a reduction in property because the material is easily deteriorated. For example, pentacene described in Cited Document 1 is oxidized and has a reduction in electrical property, as shown below.

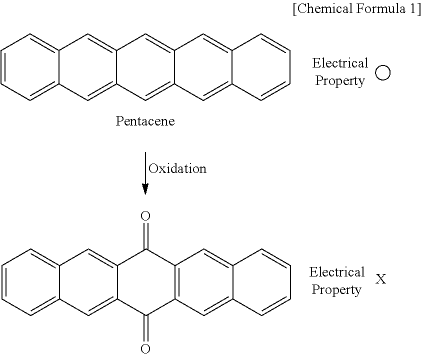

Further, a condensed compound (such as pentacene), DHDAP described in Non-patent Literature 1, and the like are low in solubility. For this reason, in order to form a semiconductor layer with the use of such a material, it is generally necessary to employ an evaporation method which is a vacuum process. This increases a production cost.

Furthermore, although Patent Literature 3 describes a condensed polycyclic aromatic heterocyclic compound used in an organic light emitting device, there has been strong demand for a novel organic semiconductor material which can be used in various semiconductor devices.

In view of the problems, an object of the present invention is to provide a novel compound that can be used as an organic semiconductor material.

### Solution to Problem

The inventors of the present invention found, as a result of diligent study in view of the problems, that a compound in which an aliphatic hydrocarbon group is introduced in a central nitrogen atom of a dihydrodiazapentacene skeleton has high oxidation resistance and excellent solubility.

That is, a compound of the present invention is represented by the following formula (1):

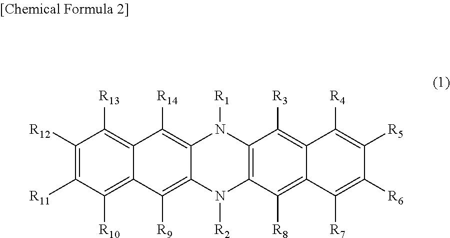

(where: R₁ and R₂ represent, independently, a substitutable C₁ to C₂₀ aliphatic hydrocarbon group; and R₃ through R₁₄ represent, independently, one of a hydrogen atom, a halogen atom, a substitutable C₁ to C₂₀ aliphatic hydrocarbon group, and a substitutable aromatic hydrocarbon group).

Further, a field effect transistor of the present invention, includes an organic semiconductor layer including any one of the aforementioned compounds of the present invention.

Furthermore, a method of the present invention, for producing a field effect transistor including an organic semiconductor layer containing a compound recited in any one of the aforementioned compounds of the present invention, includes the step of: forming the organic semiconductor layer with the use of a composition containing the compound by use of one of a dipping method, a spin coat method, a casting method, an ink-jet method, and a print method, the composition containing at least one selected from the group consisting of toluene, chlorobenzene, dichlorobenzene, trichlorobenzene, dichloromethane, and chloroform.

Moreover, a solar cell of the present invention includes a p-type semiconductor layer containing any one of the aforementioned compounds of the present invention.

Further, a method of the present invention, for producing a solar cell including a p-type semiconductor layer containing any one of the aforementioned compounds of the present invention, includes the step of: forming the p-type semiconductor layer with the use of a composition containing the compound by use of one of a dipping method, a spin coat method, a casting method, an ink-jet method, and a print method, the composition including at least one selected from the group consisting of toluene, chlorobenzene, dichlorobenzene, trichlorobenzene, dichloromethane, and chloroform.

Furthermore, a solar cell of the present invention may include an organic semiconductor layer containing a p-type semiconductor material and an n-type semiconductor material, the p-type semiconductor material containing any one of the aforementioned compounds of the present invention.

Moreover, a composition of the present invention, for an organic semiconductor layer of a field effect transistor includes any one of the aforementioned compounds of the present invention.

Further, a composition of the present invention, for a p-type semiconductor of a solar cell includes any one of the aforementioned compounds of the present invention.

Furthermore, a composition of the present invention, for an organic semiconductor layer of a solar cell includes: a p-type semiconductor material; and an n-type semiconductor material, the p-type semiconductor material containing any one of the aforementioned compounds of the present invention.

### Advantageous Effects of Invention

According to the present invention, it is possible to provide a novel compound that can be used as an organic semiconductor material.

Additional objects, features, and strengths of the present invention will be made clear by the description below. Further, the advantages of the present invention will be evident from the following explanation in reference to the drawings.

### BRIEF DESCRIPTION OF DRAWINGS

FIG. 1 is a view showing how an absorption intensity of a solution of Compound 1 changes from a time immediately after preparation of the solution to a time 1 week after the preparation.

FIG. 2 is a cross-sectional view illustrating a main part of a field effect transistor to which the present invention is applicable.

FIG. 3 Each of (a) through (e) of FIG. 3 is a cross-sectional view illustrating a step of a process of producing a field effect transistor to which the present invention is applicable.

FIG. 4 is a graph showing a gate voltage (Vg)-drain current (Id) property of an organic thin-film transistor in accordance with one example of the present invention.

FIG. 5 is a cross-sectional view illustrating a main part of a field effect transistor of another example, to which the present invention is applicable.

FIG. 6 Each of (a) through (d) of FIG. 6 is a cross-sectional view illustrating a step of a process of producing a field effect transistor of another example, to which the present invention is applicable.

FIG. 7 is a cross-sectional view illustrating a main part of a field effect transistor of another example, to which the present invention is applicable.

FIG. 8 Each of (a) through (f) of FIG. 8 is a cross-sectional view illustrating a step of a process of producing a field effect transistor of another example, to which the present invention is applicable.

FIG. 9 is a cross-sectional view illustrating a main part of a field effect transistor of another example, to which the present invention is applicable.

FIG. 10 Each of (a) through (e) of FIG. 10 is a cross-sectional view illustrating a step of a process of producing a field effect transistor of another example, to which the present invention is applicable.

### DESCRIPTION OF EMBODIMENTS

### [1. Compound of the Present Invention]

A compound of the present invention is represented by the aforementioned formula (1). That is, the compound of the present invention has a dihydrodiazapentacene skeleton.

In the aforementioned formula (1), R₁ and R₂ represent, independently, a C₁ to C₂₀ aliphatic hydrocarbon group. The aliphatic hydrocarbon group may be either saturated or unsaturated, and may be a linear, branched, or cyclic aliphatic hydrocarbon group. Here, examples of a saturated or unsaturated and liner or branched aliphatic hydrocarbon group encompass a methyl group, an ethyl group, a propyl group, an isopropyl group, an n-butyl group, an isobutyl group, an aryl group, a t-butyl group, an n-pentyl group, an n-hexyl group, an n-octyl group, an n-decyl group, an n-dodecyl group, an n-stearyl group, and an n-butenyl group. Further, examples of a cyclic aliphatic hydrocarbon group encompass a cycloalkyl group. Examples of the cycloalkyl group encompass a C₃ to C₁₂ cycloalkyl group, such as a cyclohexyl group, a cyclopentyl group, an adamantyl group, and a norbornyl group.

In the aforementioned formula (1), the aliphatic hydrocarbon group represented by R₁ and R₂ may be substituted. Examples of a substituent group encompass a halogen atom, a hydroxyl group, a mercapto group, a nitro group, an alkoxyl group, an alkyl-substituted amino group, an aryl-substituted amino group, an unsubstituted amino group, an aryl group, and an acyl group.

Note that R₁ and R₂ may be either identical with each other or different from each other.

In the aforementioned formula (1), R₃ through R₁₄ represent, independently, one of a hydrogen atom, a halogen atom, a C₁ to C₂₀ aliphatic hydrocarbon group, and an aromatic hydrocarbon group.

Examples of the aliphatic hydrocarbon group represented by any one of R₃ through R₁₄ may be identical with those of the aliphatic hydrocarbon group represented by R₁ and R₂.

Examples of the aromatic hydrocarbon group represented by any one of R₃ through R₁₄ encompass a phenyl group, a pyridyl group, a furyl group, a thienyl group, a selenothienyl group, a naphthyl group, and an anthryl group.

The aliphatic hydrocarbon group or the aromatic hydrocarbon group, represented by any one of R₃ through R₁₄, may be substituted. Examples of a substituent group may be identical with those of the aliphatic hydrocarbon group represented by R₁ and R₂.

According to the compound of the present invention, it is preferable that, in the aforementioned formula (1), R₁ and R₂ represent, independently, a substitutable C₆ to C₂₀ aliphatic hydrocarbon group, and R₃ through R₁₄ represent a hydrogen atom. Further, it is preferable that R₁ and R₂ represent an n-hexyl group. That is, it is preferable that the compound of the present invention is represented by the following formula (2).

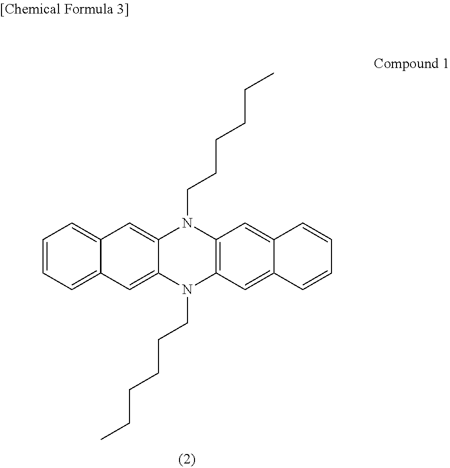

### [2. Method of Present Invention, for Producing Compound]

As an example of a method of the present invention, for producing a compound, a method of synthesizing Compound 1 represented by the above formula (2) is described in the following Example 2. The method of the present invention, for synthesizing a compound, is not limited to the method of synthesizing a compound, described in the following Example 2. Note, however, that various compounds represented by the aforementioned formula (1) can be synthesized in accordance with the method of synthesizing a compound, described in the following Example 2. For example, the compound represented by the aforementioned formula (1) can be synthesized by (i) selecting a starting material in accordance with a target compound, and (ii) using the method of synthesizing a compound, described in the following Example 2.

Further, as another example of the method of the present invention, for producing a compound, there is such a method that, in a case where Compound 1 is synthesized from Intermediate 1 in the method of synthesizing a compound, described in the following Example 2, butyllithium (BuLi) is used in place of sodium hydride (NaH).

### [3. Use of Compound of the Present Invention]

The compound of the present invention can be used as a semiconductor layer material of an organic electronic device (such as a field effect transistor and a solar cell). That is, according to the present invention, it is possible to provide a field effect transistor and a solar cell, each of which includes a semiconductor layer containing the aforementioned compound. Further, according to the present invention, it is possible to provide a composition for an organic semiconductor layer of a field effect transistor, and a composition for a p-type semiconductor layer of a solar cell, each of which contains the aforementioned compound.

### <3-1. Field Effect Transistor>

One embodiment of a field effect transistor of the present invention is described below.

### (Arrangement of Field Effect Transistor)

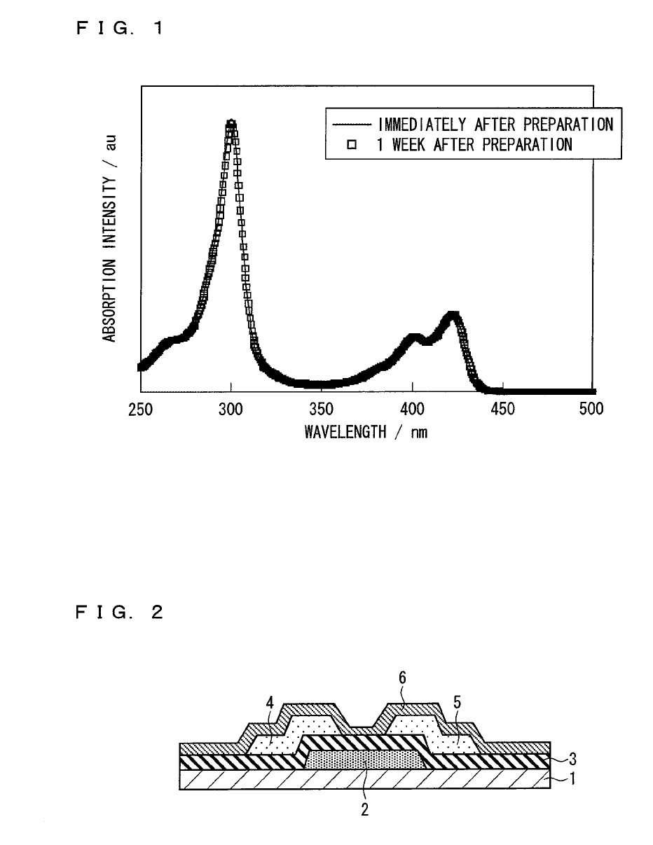

First, the following description deals with an arrangement of a field effect transistor in accordance with the present embodiment, with reference to FIG. 2. FIG. 2 is a cross-sectional view illustrating a main part of the field effect transistor to which the present invention is applicable.

The field effect transistor is such that (i) a gate electrode 2, a gate insulating film 3, and a source electrode 4/drain electrode 5 are provided on a substrate 1 in this order, and (ii) an organic semiconductor layer 6 is provided to cover the source electrode 4/drain electrode 5 (see FIG. 2). That is, the field effect transistor includes the substrate 1, the gate electrode 2 provided on the substrate 1, the gate insulating film 3 provided to cover the gate electrode 2, the source electrode 4 provided on the gate insulating film 3, the drain electrode 5 provided on the gate insulating film 3, and the organic semiconductor layer 6 provided to cover the source electrode 4 and the drain electrode 5. In other words, the field effect transistor has an arrangement of a bottom-gate/bottom-contact transistor.

An example of producing the field effect transistor having the arrangement described above is described in the following Example 3. The field effect transistor of the present invention can be produced in accordance with the method described in the following Example 3. Note that the field effect transistor to which the present invention is applicable is not limited to the one described above. For example, the field effect transistor can have any one of the following arrangements: (1) an arrangement in which (i) a gate electrode, a gate insulating film, and a source electrode/drain electrode are provided on a substrate in this order, and (ii) an organic semiconductor layer is provided on the gate insulating film so as to be between the source electrode and the drain electrode, (2) an arrangement in which (i) an organic semiconductor layer and a source electrode/drain electrode are provided on a substrate in this order, and (ii) a gate insulating film and a gate electrode are provided on the organic semiconductor layer in this order so as to be between the source electrode and the drain electrode, (3) an arrangement in which a gate electrode, a gate insulating film, an organic semiconductor layer, and a source electrode/drain electrode are provided on a substrate in this order, and (4) an arrangement in which (i) a source electrode/drain electrode is provided on a substrate, (ii) an organic semiconductor layer and a gate insulating film are provided in this order so as to cover the source electrode/drain electrode, and (iii) a gate electrode is provided on the gate insulating film.

That is, the field effect transistor of the present invention can have an arrangement illustrated in FIG. 5, for example. FIG. 5 is a cross-sectional view illustrating a main part of a field effect transistor of another example, to which the present invention is applicable. The field effect transistor illustrated in FIG. 5 includes a gate electrode, a gate insulating film 3 provided to cover the gate electrode 2, a source electrode 4, a drain electrode 5, and an organic semiconductor layer 6 so that (i) the organic semiconductor layer 6 is provided on the gate insulating film 3 and (ii) the source electrode 4 and the drain electrode 5 are provided on the organic semiconductor layer 6 so as to be in contact with the organic semiconductor layer 6. In other words, the field effect transistor illustrated in FIG. 5 has a top-contact structure.

Here, a film property of the organic semiconductor layer 6 may be influenced by a layer (base layer) provided below the organic semiconductor layer 6. In a case of the bottom-contact structure illustrated in FIG. 2, the organic semiconductor layer 6 is constituted by (i) a first part which is provided on the gate insulating film 3 and (ii) a second part which is provided on the source electrode 4 and the drain electrode 5. Accordingly, the first and second parts may become different from each other in film property. In this case, the film property of the entire organic semiconductor layer 6 may be reduced.

On the other hand, in a case of the top-contact structure illustrated in FIG. 5, the entire organic semiconductor layer 6 is provided on the gate insulating film 3. Accordingly, it is possible to (i) form the organic semiconductor layer 6 which has a uniform film property, and therefore (ii) obtain a field effect transistor which has a stable semiconductor property.

Further, in a case where the source electrode 4 and the drain electrode 5 are provided on the gate insulating film 3, there is a risk that, in formation of the source electrode 4 and the drain electrode 5, the gate insulating film 3 might be damaged or might have a residue. However, in the case of the top-contact structure, there is no risk that the gate insulating film 3 might be damaged or might have a residue. Accordingly, it is possible to form an interface between the organic semiconductor layer 6 and the gate insulating film 3 successfully while not being influenced by the aforementioned damage and the like.

The following description deals with each of structural elements of the field effect transistor of the present invention more specifically.

### (Organic Semiconductor Layer 6)

The organic semiconductor layer 6 contains the aforementioned compound of the present invention. The organic semiconductor layer 6 may be formed in such a manner that a composition containing the compound of the present invention is applied. For example, the organic semiconductor layer 6 can be formed in such a manner that a composition for an organic semiconductor layer (later described) is subjected to a low-cost thin-film formation method such as a dipping method, a casting method, a spin coat method, a print method employing an inkjet method, or the like. That is, a method of the present embodiment, for producing a field effect transistor, can include the step of forming the organic semiconductor layer 6 with the use of a composition containing any of the aforementioned compounds of the present invention (preferably the composition for an organic semiconductor layer (later described)) by use of one of the dipping method, the spin coat method, the casting method, the inkjet method, or the print method.

Further, the organic semiconductor layer 6 may be formed in such a manner that a compound is evaporated by use of a vacuum evaporation method or the like.

Furthermore, the compound of the present invention has a high oxidation resistance (later described). Accordingly, with the organic semiconductor layer 6 containing the compound of the present invention, it is possible to provide an organic semiconductor element which can operate stably in the atmosphere.

Further, it is preferable that the organic semiconductor layer 6 is provided on a hydrophilic film. The hydrophilic film is a film whose surface has a hydrophilic property. For example, in a case where the organic semiconductor layer 6 is formed on the gate insulating film 3, the hydrophilic film can be provided on the gate insulating film 3 or the gate insulating film 3 itself can serve as the hydrophilic film. That is, the gate insulating film 3 can be arranged so that a surface of the gate insulating film 3 serves as a hydrophilic film having a hydrophilic property. Furthermore, in a case where the organic semiconductor layer 6 is provided on the source electrode 4 and the drain electrode 5 (for example, in the case of the bottom-gate/bottom-contact structure), the hydrophilic film may be provided on the source electrode 4 and the drain electrode 5. In this case, it is preferable to provide the organic semiconductor layer 6 on such a hydrophilic film.

In the case where the hydrophilic film is provided on the gate insulating film 3, or in the case where the gate insulating film 3 serves as the hydrophilic film, the hydrophilic film may be made from a metallic oxide insulating material, a hydrophilic polymer, or the like. Examples of the metallic oxide insulating material encompass a silicon oxide film and an aluminum oxide film. Further, examples of the hydrophilic polymer encompass polyethylene glycol, polyacrylic acid, and polyvinyl alcohol.

Furthermore, in a case where the hydrophilic film is provided on the source electrode 4 and the drain electrode 5, the hydrophilic film may be a film whose surface has a hydrophilic group, for example. Examples of the hydrophilic group encompass a hydroxyl group, an amino group, a carboxyl group, a sulfonate group, and a phosphate group. Such a hydrophilic film can be formed, for example, in such a manner that a surface of the source electrode 5 and a surface of the drain electrode 5 are modified through surface treatment (hereinafter, referred to as “surface modification”, in some cases). How to carry out the surface modification will be described in the following examples.

By providing the hydrophilic film, it is possible to form the organic semiconductor layer 6 uniformly. Particularly, in a case where the organic semiconductor layer 6 is formed by application of the composition for an organic semiconductor layer (later described), the provision of the hydrophilic film realizes a significant advantageous effect.

### (Gate Electrode 2, Source Electrode 4/Drain Electrode 5)

A material of the gate electrode 2 is not particularly limited, and may be a known material of a general gate electrode. Specifically, examples of the material of the gate electrode 2 encompass a metal material having a low resistance (such as gold, platinum, silver, copper, aluminum, tantalum, and doped silicon) and an organic conductive material (such as PEDOT/PSS).

As a material of the source electrode 4/drain electrode 5, it is possible to employ a material which is substantially the same as the composition for an organic semiconductor layer in highest occupied molecular orbital (HOMO) level, or a material which is substantially the same as the composition for an organic semiconductor layer in lowest unoccupied molecular orbital (LUMO) level. Examples of the material which is substantially the same as the composition for an organic semiconductor layer in HOMO level encompass a metal having a relatively high work function (such as gold, platinum, silver, and an alloy containing any of these), a transparent oxide conductive material (such as ITO and zinc oxide (ZnO)), and an organic conductive material (such as PEDOT: PSS). On the other hand, examples of the material which is substantially the same as the composition for an organic semiconductor layer in LUMO level encompass a metal having a relatively low work function (such as aluminum, titanium, an alkali metal, and an alloy containing any one of these).

Further, a surface of the source electrode 4/drain electrode 5 may be modified with organic molecules or the like.

A film thickness of each of the electrodes is not particularly limited, and may be equal to a film thickness of an electrode used in a general transistor (e.g., in a case of a metallic electrode, the film thickness may be in a range of 30 nm to 200 nm). It is preferable to adjust the film thickness appropriately, if necessary. Examples of a method of preparing each of the electrodes encompass an evaporation method, a sputtering method, and a coating method. It is preferable to select such a preparation method appropriately in accordance with a material thus used.

### (Gate Insulating Film 3)

As a material of the gate insulating film 3, it is preferable to select a material which (i) has a high dielectric constant and (ii) is not likely to have a defect of a pin hole in formation of a thin film. In a case where the material has a high dielectric constant, it is possible to cause a threshold voltage of a field effect transistor to be low. Further, it is preferable that a film thickness of the gate insulating film 3 is small. In a case where the gate insulating film 3 is thin, it is possible to reduce a threshold voltage of the field effect transistor. Furthermore, in a case where the material is not likely to have a defect of a pin hole in formation of the thin film, the gate insulating film 3 would not be reduced in function. It becomes therefore possible to obtain a field effect transistor having an excellent function.

Examples of such a material encompass an inorganic insulating film (such as a silicon oxide film, silicon nitride film, a tantalum pentoxide film, and an aluminum oxide film), and an organic insulating film (such as a polyimide film, a parylene membrane, and a polyvinyl phenol membrane).

It is preferable that (i) the gate insulating film 3 has such a film thickness that an electrostatic capacity is large per unit area and (ii) the film thickness is appropriately determined in accordance with a specific permittivity, an insulation property, and the like of the material of the gate insulating film 3. The film thickness of the gate insulating film 3 is preferably in a range of 50 nm to 300 nm, for example. With the arrangement, it is possible to reduce the threshold voltage of the field effect transistor.

Further, in a case where a silicon oxide film, a silicon nitride film, or the like is used as the gate insulating film 3, it is preferable that a surface of the gate insulating film 3, which surface is in contact with the organic semiconductor layer, is treated with a silane coupling agent or the like. This can cause a grain size of crystals of the organic semiconductor layer to be large, which organic semiconductor layer is in contact with the gate insulating film. It is therefore possible to improve mobility of the organic semiconductor element.

Examples of a method of preparing the gate insulating film 3 encompass an evaporation method, a sputtering method, and a coating method. It is preferable to select appropriately the method of preparing the gate insulating film 3 in accordance with the material thus used.

### <3-2. Composition for Organic Semiconductor Layer of Field Effect Transistor>

A composition of the present invention, for an organic semiconductor layer of a field effect transistor, is a composition used as a material of the organic semiconductor layer of the field effect transistor, and contains the aforementioned compound of the present invention. The composition for the organic semiconductor layer preferably contains the compound of the present invention in an amount in a range of 0.5% by weight to 5% by weight. Furthermore, examples of a solvent used with the compound encompass chloroform, toluene, chlorobenzene, dichlorobenzene, trichlorobenzene, and dichloromethane. Among these, toluene is preferably used as the solvent.

By employing such a composition for an organic semiconductor layer, it becomes possible to form the organic semiconductor layer of the field effect transistor by a low-cost film formation method, such as a print method, a casting method, and a spin coat method. Accordingly, it becomes unnecessary to use a vacuum apparatus or the like. It is therefore possible to reduce a production cost of the field effect transistor. Note that the compound of the present invention can be dissolved into the solvent, so that the aforementioned composition for an organic semiconductor layer can be prepared easily.

### <3-3. Solar Cell>

Next, the following description deals with a solar cell of the present invention.

### (Arrangement of Solar Cell)

One embodiment of the solar cell (organic solar cell) of the present invention is, for example, such that (i) a solar cell includes a pair of an anode electrode and a cathode electrode, a p-type semiconductor layer, and an n-type semiconductor layer, and (ii) the p-type semiconductor layer and the n-type semiconductor layer are subjected to PN junction, and are provided between the anode electrode and the cathode electrode.

The p-type semiconductor layer contains the aforementioned compound of the present invention. The p-type semiconductor layer may be formed by application of the composition containing the compound of the present invention. For example, the p-type semiconductor layer can be formed in such a manner that a composition for a p-type semiconductor layer (later described) is subjected to a low-cost thin-film formation method such as a dipping method, a casting method, a spin coat method, and an ink-jet method. That is, a method of the present invention, for producing a solar cell, includes the step of forming the p-type semiconductor layer with the use of a composition containing any of the aforementioned compounds in accordance with the present invention (preferably the composition for a p-type semiconductor layer (Later described)) by use of any one of the dipping method, the spin coat method, casting method, the ink-jet method, and the print method.

Further, the p-type semiconductor layer may be formed by evaporating a compound by a vacuum evaporation method or the like.

Furthermore, it is preferable that the p-type semiconductor layer is formed on a hydrophilic film. Examples of the hydrophilic film may be identical with those of the hydrophilic film used for the field effect transistor, described above. In this case, it is possible to obtain effects similar to those of the hydrophilic film used for the field effect transistor, described above.

Note that the compound of the present invention has a high oxidation resistance as described below. Accordingly, with the use of the p-type semiconductor layer containing the compound of the present invention, it is possible to provide an organic semiconductor element which can operate stably in the atmosphere.

Examples of a material of the n-type semiconductor layer encompass fullerene (or a fullerene derivative) and fluorinated phthalocyanine. Further, examples of the anode electrode encompass ITO which serves as a transparent electrode, and PEDOT: PSS. Furthermore, examples of a material of the cathode electrode encompass silver and aluminum.

Moreover, another embodiment of the solar cell of the present invention is such that a solar cell includes an organic semiconductor layer containing a p-type semiconductor material and an n-type semiconductor material. That is, the solar cell of the present invention may be an organic thin-film solar cell, for example. The p-type semiconductor material contains the compound of the present invention. The organic semiconductor layer can be formed, for example, with the use of the composition for an organic semiconductor layer of a solar cell (later described) by a preparation method identical with that of the p-type semiconductor layer described above.

<3-4. Composition for p-Type Semiconductor Layer>

A composition of the present invention, for a p-type semiconductor layer of a solar cell, is a composition which is used as a material of the p-type semiconductor layer of the solar cell having the arrangement described above, and contains the compound of the present invention. It is preferable that the composition for a p-type semiconductor layer contains the compound of the present invention in an amount in a range of 0.5% by weight to 5% by weight. Further, examples of a solvent used with the compound encompass chloroform, toluene, chlorobenzene, dichlorobenzene, trichlorobenzene, and dichloromethane. Among these, toluene is preferably used as the solvent.

By employing such a composition for a p-type semiconductor layer, it is possible to form the p-type semiconductor layer of the solar cell by use of a low-cost film formation method such as a print method, a casting method, and a spin coat method. Accordingly, it becomes unnecessary to use a vacuum apparatus or the like. It is therefore possible to reduce a production cost of the solar cell. Note that, the compound of the present invention can be dissolved into a solvent, so that the aforementioned composition for a p-type semiconductor layer can be prepared easily.

### <3-5. Composition for Organic Semiconductor Layer of Solar Cell>

A composition for an organic semiconductor layer of a solar cell is a composition for forming an organic semiconductor layer of the solar cell described above, and contains a p-type semiconductor material and an n-type semiconductor material. The p-type semiconductor material may be either the one containing the composition of the present invention, or the composition of the present invention itself. Examples of the n-type semiconductor material may be identical with those of the n-type semiconductor layer described above.

It is preferable that the composition for an organic semiconductor layer contains the p-type semiconductor material in an amount in a range of 0.5% by weight to 5% by weight. Further, it is preferable that the composition for an organic semiconductor layer contains the n-type semiconductor material in an amount in a range of 0.5% by weight to 5% by weight. Furthermore, examples of a solvent used with the compound encompass chloroform, toluene, chlorobenzene, dichlorobenzene, trichlorobenzene, and dichloromethane. Among these, toluene is preferably used as the solvent.

With the use of such a composition for an organic semiconductor layer, it is possible to form the organic semiconductor film of the solar cell by use of a low-cost film formation method as described above. It becomes therefore possible to reduce a production cost of the solar cell.

### [4. Additional Matters]

Note that the compound of the present invention is preferably arranged such that, in the formula (1), R₁ and R₂ represent, independently, a substitutable C₆ to C₂₀ aliphatic hydrocarbon group, and R₃ through R₁₄ represent a hydrogen atom.

Further, in the formula (1), R₁ and R₂ may represent an n-hexyl group.

Furthermore, the field effect transistor of the present invention is preferably arranged such that the organic semiconductor layer is provided on a hydrophilic film.

Moreover, the field effect transistor of the present invention is preferable arranged such that (i) the field effect transistor includes a gate electrode, a gate insulating film, a source electrode, and a drain electrode, the gate electrode being covered with the gate insulating film, (ii) the organic semiconductor layer is provided on the gate insulating film, and (iii) the source electrode and the drain electrode are provided on the organic semiconductor layer so as to be in contact with the organic semiconductor layer.

Further, the field effect transistor of the present invention is preferably arranged such that (i) the field effect transistor includes a gate electrode, a gate insulating film, a source electrode, and a drain electrode, the gate electrode being covered with the gate insulating film, the source electrode and the drain electrode being provided on the gate insulating film, and (ii) the organic semiconductor layer is provided so as to cover the source electrode and the drain electrode.

Furthermore, the field effect transistor of the present invention is preferably arranged such that the organic semiconductor layer is formed by application of any one of the aforementioned compounds of the present invention.

Moreover, the field effect transistor of the present invention is preferably arranged such that the organic semiconductor layer is formed by evaporation of any one of the aforementioned compounds of the present invention.

Further, the method of the present invention, for producing a field effect transistor, is preferably arranged such that the composition contains toluene.

Furthermore, the solar cell of the present invention is preferably arranged such that the p-type semiconductor layer is provided on a hydrophilic film.

Moreover, the solar cell of the present invention is preferably arranged such that the p-type semiconductor layer is formed by application of any one of the aforementioned compounds of the present invention.

Further, the solar cell of the present invention is preferably arranged such that the p-type semiconductor layer is formed by evaporation of any one of the aforementioned compounds of the present invention.

Furthermore, the method of the present invention, for producing a solar cell, is preferably arranged such that the composition contains toluene.

Moreover, the compound of the present invention may be represented by the following formula (4):

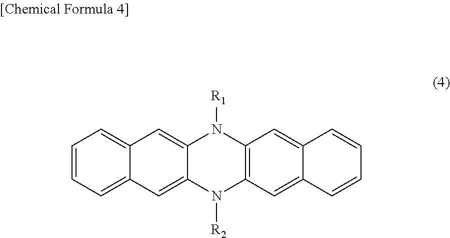

(where: R₁ and R₂ represent, independently, a substitutable C₁ to C₂₀ aliphatic hydrocarbon group).

The present invention is not limited to the description of the embodiments above, but may be altered by a skilled person within the scope of the claims. An embodiment based on a proper combination of technical means disclosed in different embodiments is encompassed in the technical scope of the present invention.

The following description deals with examples so as to describe embodiments of the present invention more specifically. As a matter of course, the present invention is not limited to the following examples, and details can be modified variously.

### EXAMPLES

### Example 1

In Example 1, molecular orbital calculation was carried out for Compound 2 represented by the following chemical formula (3) by a DFT method (B3LYP6-31G*), so as to calculate a level of a highest occupied molecular orbital (HOMO) and a level of a lowest unoccupied molecular orbital (LUMO).

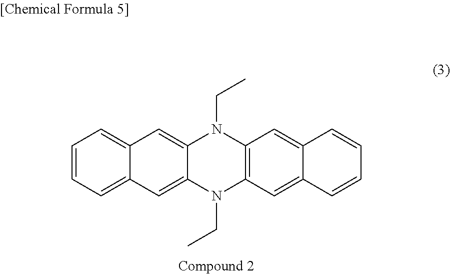

A result of the calculation is shown in the following Table 1, with results obtained with the use of the following pentacene and DHDAP.

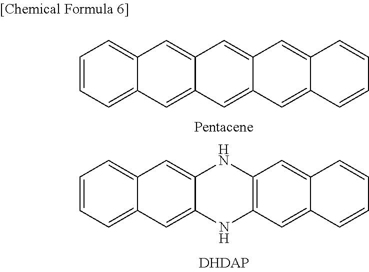

TABLE 1
Molecular orbital calculation by DFT method
(B3LYP6-31G*) (unit: eV)

| Target | | | |
|---|---|---|---|
| molecule | HOMO | LUMO | LUMO − HOMO |
| Compound 2 | −4.55 | −0.91 | 3.64 |
| Pentacene | −4.60 | −2.39 | 2.21 |
| DHDAP | −4.62 | −0.96 | 3.66 |

As a result of the molecular orbital calculation, Compound 2, pentacene, and DHDAP showed a HOMO level of approximately −4.6 eV, and there was no significant difference between Compound 2, pentacene, and DHDAP in HOMO level. Accordingly, it seemed that Compound 2 had an ionization potential substantially equal to that of pentacene.

It seems that, in a case where Compound 2 having the above HOMO level is used as an organic semiconductor layer of a field effect transistor employing a source/gate electrode made from, for example, ITO (work function: approximately 4.8 eV) or gold (work function: approximately 5 eV), it would become easy to carry out hole (electron hole) injection.

### Example 2

Next, in Example 2, Compound 1 represented by the aforementioned formula (2) was synthesized in accordance with the following reaction formula (1). The following description deals with how to synthesize Compound 1.

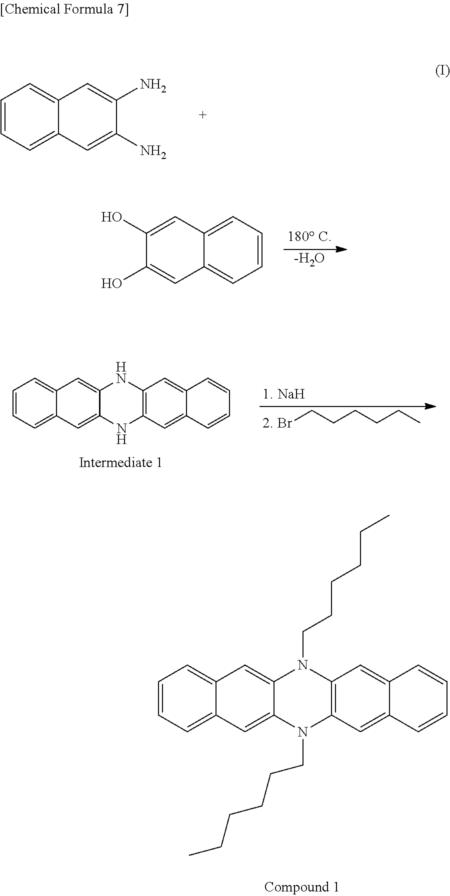

### (Synthesis of Intermediate 1)

First, DHDAP was synthesized as a synthetic intermediate (Intermediate 1) of Compound 1.

In a nitrogen gas, 2,3-dihydroxy naphthalene (0.93 g, 5.8 mmol, produced by Wako Pure Chemical Industries, Ltd.) and 2,3-naphthalene diamine (0.93 g, 5.8 mmol, produced by Wako Pure Chemical Industries, Ltd.) were sufficiently mixed with each other, and then were heated at 180° C. for 1 hour. The resultant product was washed with acetone, and was recrystallized from DMF in the nitrogen gas, so that 1.08 g of Intermediate I was obtained (yield: 66%).

### (Synthesis of Compound 1)

Next, Compound 1 was synthesized from Intermediate 1.

Into an anhydrous DMF (15 ml) solution of Intermediate 1 (0.42 g, 1.5 mmol), sodium hydride (60%, 0.13 g, 3.1 mmol) was added little by little. The resultant solution was left at room temperature for 2 hours. After that, 1-bromohexane (0.5 g, 3 mmol) was added to the solution, and the solution was agitated for further 2 hours. A solvent was removed away under reduced pressure, and a residue was extracted with methylene chloride. The residue from which the solvent was distilled away was recrystallized from hexane so that 0.28 g of Compound 1 was obtained (yield: 41%). Compound 1 was a yellow crystal, and had no hydroscopic property. Further, Compound 1 was a stable compound without any deterioration in the atmosphere.

An absorption spectrum (absorption peak wavelength λmax, molar absorbance coefficient c) of Compound 1 thus synthesized was measured. Further, a HOMO level (ionization potential) of Compound 1 was measured by photoelectric spectroscopy in the atmosphere (SC-2). The following Table 2 shows a result of the measurement with a result obtained with the use of pentacene.

TABLE 2
Results of measurement of absorption spectrum

| | | Photoelectric |
|---|---|---|
| Target | Absorption spectrum | spectroscopy |
| molecules | λ max(ε) in CH₂CL₂ | HOMO (eV) |
| Compound 1 | 422 nm | −4.89 |
| | (log ε = 4.58) | |
| Pentacene | 575 nm | −5.0 |

Note that, for the result of pentacene shown in Table 2, the absorption spectrum described in Reference Document 1 (J. Am. Chem. Soc. 2007, 129, 2225) was used, and the HOMO level described in Reference Document 2 (Jpn. J. Appl. Phys. 2005, 44, 561) was used.

An absorption wavelength of Compound 1 was significantly shifted toward a short wavelength side as compared with pentacene. That is, a HOMO-LUMO gap of Compound 1 was significantly greater than that of pentacene. Further, the HOMO level of Compound 1 measured by photoelectric spectroscopy in the atmosphere was substantially equal to that of pentacene. These results well matched a pattern of the molecular orbital calculation of Example 1. Accordingly, the results showed that Compound 1 has an ionization potential which was substantially equal to that of pentacene.

Next, stability of Compound 1 against aerial oxidation was evaluated. A solution of Compound 1 was prepared with the use of air-saturated methylene chloride as a solvent, and was left in a dark place. The absorption spectrum was measured during a time period from immediately after the preparation to 1 week after the preparation, so as to trace how an absorption intensity of the solution of Compound 1 was changed during the above time period (see Reference Document 1 described above).

FIG. 1 shows (i) the absorption intensity of the solution of Compound, obtained immediately after the preparation and (ii) the absorption intensity of the solution of Compound 1, obtained 1 week after the preparation. As to Compound 1, there was no change in absorption intensity from the time immediately after the preparation to the time 1 week after the preparation (left in the dark place). Note that, as to pentacene, it was reported that the absorption intensity was substantially eliminated in a case where pentacene was left for 24 hours on the above condition (see Reference Document 1). Accordingly, the results showed that Compound 1 was significantly higher than pentacene in stability against aerial oxidation.

As described above, it was shown that the compound of the present invention has a sufficiently high HOMO level, and therefore retains an excellent electric property. Further, as compared with pentacene, the compound of the present invention has a higher oxidation resistance, and is stable in the atmosphere. Accordingly, by employing the compound of the present invention as a semiconductor material, it becomes possible to provide a semiconductor device which has a stable and excellent electric property.

Further, Compound 1 thus synthesized had an excellent resolvability with respect to a solvent. Accordingly, in a case where Compound 1 is used as a composition for semiconductor layer of a semiconductor device, it is possible to select a low-cost film formation method (such as a coating method) in formation of the semiconductor layer.

Furthermore, the compound of the present invention can be synthesized easily from a commercial reagent, as in the aforementioned method in which Compound 1 was synthesized.

### Example 3

In Example 3, an organic thin-film transistor having the same arrangement as that of a field effect transistor illustrated in FIG. 2 was produced, and properties of the organic thin-film transistor were evaluated. That is, the organic thin-film transistor produced in the present example had, as described above, an arrangement in which (i) a gate electrode 2, a gate insulating film 3, and a source electrode 4/drain electrode 5 were provided on a substrate 1 in this order, and (ii) an organic semiconductor layer 6 was provided so as to cover the source electrode 4/drain electrode 5.

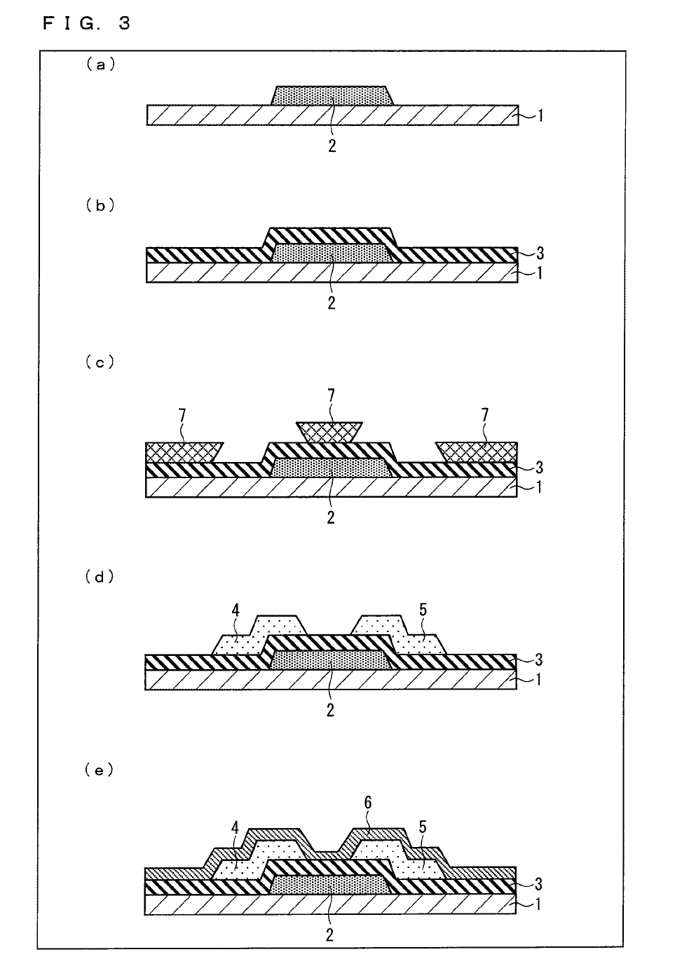

The following description deals with a method of producing an organic thin-film transistor in accordance with the present example with reference to (a) through (e) of FIG. 3. Each of (a) through (e) of FIG. 3 is a cross-sectional view illustrating a step of a process of producing a field effect transistor to which the present invention is applicable.

First, the gate electrode 2 was provided on the substrate 1 (see (a) of FIG. 3). As the substrate 1, a glass substrate (Eagle 2000, thickness: 0.5 mm, manufactured by Corning Incorporated) was used. Further, as the gate electrode 2, an AlSi alloy in which 10% of silicon (Si) was added to aluminum (Al) was used. By use of a sputtering method employing a target metal made from the AlSi alloy, a metallic film was formed on the substrate 1. The metallic film was made from the AlSi alloy and had a film thickness of 40 nm. Next, the metallic film made from the AlSi alloy was subjected to photolithography and etching so as to be in a desired pattern. The gate electrode 2 illustrated in (a) of FIG. 3 was thus formed.

Next, the gate insulating film 3 was formed as illustrated in (b) of FIG. 3. As the gate insulating film 3, a silicon oxide film (SiO₂) was used. By use of the sputtering method, a silicon oxide film having a film thickness of 300 nm was formed on the gate electrode 2. The gate insulating film 3 was thus formed.

Next, in order to form the source electrode 4 and the drain electrode 5 by a liftoff technique, a photoresist film 7 having an opening section 7 was formed (see (c) of FIG. 3). As the photoresist film 7, a negative photoresist (ZPN1150, manufactured by ZEON CORPORATION) for a liftoff process was used. By use of the spin coat method, a resist film having a film thickness of 4 μm was formed on the gate insulating film 3. Then, the resist film was subjected to an exposure process and a development process by use of photolithography. The photoresist film 7 having a desired opening section was thus formed.

Next, the source electrode 4 and the drain electrode 5 were formed (see (d) of FIG. 3). By use of a vacuum evaporation method, a contact layer (not illustrated) made from chromium (Cr) and a metallic film made from gold (Au) were formed on the photoresist film 7. Specifically, a Cr film having a film thickness of 2 nm was formed as the contact layer, and then an Au film having a film thickness of 40 nm was formed successively. After the Au film was formed, a liftoff process was carried out so as to remove away (i) the photoresist film 7 provided on the gate insulating film 3 and (ii) an unnecessary part of the Au film/Cr film formed on the photoresist film 7. In the liftoff process, the substrate was immersed in an organic solvent (such as acetone). A distance (channel length) between the source electrode 4 and the drain electrode 5 was 20 μm, and a length (channel width) of each of the electrodes facing each other was 1000 μm.

Next, the organic semiconductor layer 6 was formed (see (e) of FIG. 3). As the organic semiconductor layer 6, a composition for an organic semiconductor layer, containing Compound 1 synthesized in Example 2, was used. As the composition for an organic semiconductor layer, a solution of Compound 1 (concentration: 0.5 wt %) in which chloroform was used as a solvent was used. The solution of the composition for an organic semiconductor layer was dropped, by use of a dispenser (not illustrated), on the source electrode 4, the drain electrode 5, and the gate insulating film 3 sandwiched between the source electrode 4 and the drain electrode 5. Then, by use of a casting method in which the solution was dried slowly in a gas of saturated chloroform, the organic semiconductor layer 6 was formed. The organic semiconductor layer 6 had a film thickness of approximately 40 nm.

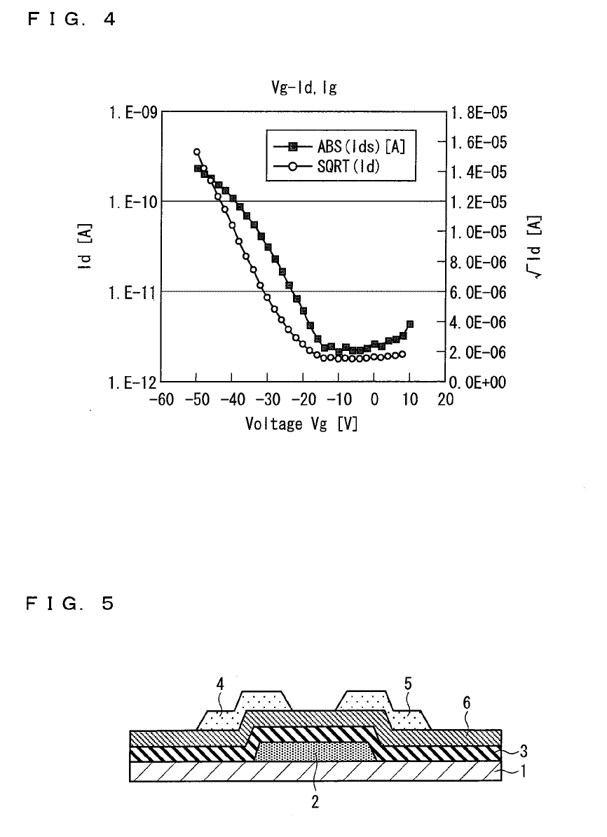

With the use of the organic thin-film transistor produced through the aforementioned process, a gate voltage (Vg)-drain current (Id) property was measured. FIG. 4 shows a result of the measurement. FIG. 4 is a graph showing a gate voltage (Vg)-drain current (Id) property of the organic thin-film transistor in accordance with one example of the present invention. The organic thin-film transistor of the present example showed an excellent transistor property, as shown in FIG. 4. Here, an electron field-effect mobility was 1.15×10⁻⁵ cm²/Vs.

### Example 4

In Example 4, an organic thin-film transistor having the same arrangement as that of a field effect transistor illustrated in FIG. 5 was produced. That is, the organic thin-film transistor produced in the present example had an arrangement in which (i) a gate electrode 2, a gate insulating film 3, and an organic semiconductor layer 6 were provided on a substrate 1 in this order so that the gate electrode was covered with the gate insulting film 3, and (ii) a source electrode 4 and a drain electrode 5 were provided on the organic semiconductor layer 6 (see FIG. 5).

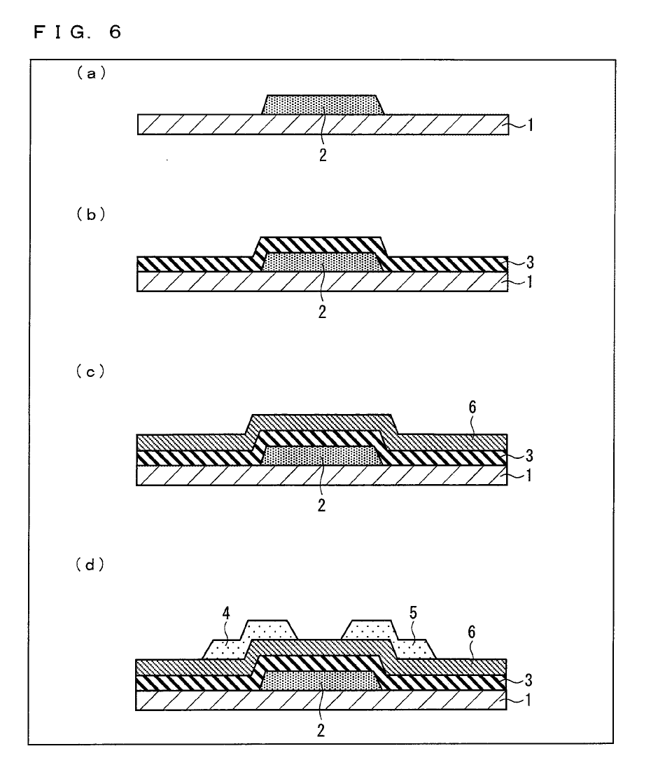

The following description deals with a method of producing an organic thin-film transistor of the present example with reference to (a) through (d) of FIG. 6. Each of (a) through (e) of FIG. 6 is a cross-sectional view illustrating a step of a process of producing a field effect transistor of another example, to which the present invention is applicable.

First, the gate electrode 2 was provided on the substrate 1 (see (a) of FIG. 6). As the substrate 1, a glass substrate (Eagle 2000, thickness: 0.5 mm, manufactured by Corning Incorporated) was used. Further, as the gate electrode 2, an AlSi alloy in which 10% of silicon (Si) was added to aluminum (Al) was used. By use of a sputtering method employing a target metal made from the AlSi alloy, a metallic film was formed on the substrate 1. The metallic film was made from the AlSi alloy and had a film thickness of 40 nm. Then, the metallic film was subjected to photolithography and etching so as to be in a desired pattern. The gate electrode 2 illustrated in (a) of FIG. 6 was thus formed.

Next, the gate insulating film 3 was formed (see (b) of FIG. 6). As the gate insulating film 3, a silicon oxide film (SiO₂) was used. By use of the sputtering method, the silicon oxide film having a film thickness of 300 nm was formed on the gate electrode 2. The gate insulating film 3 was thus formed.

Next, the organic semiconductor layer 6 was formed (see (c) of FIG. 6). As the organic semiconductor layer 6, a composition for an organic semiconductor layer, containing Compound 1 synthesized in Example 2, was used. As the composition for an organic semiconductor layer, a solution of Compound 1 (concentration: 0.5 wt %), in which toluene was used as a solvent, was used. By use of a spin coat method (the number of rotations: 1500 rpm), the organic semiconductor layer 6 was formed on the gate insulating film 3. The organic semiconductor layer 6 thus formed had a film thickness of approximately 40 nm.

Next, the source electrode 4 and the drain electrode 5 were formed (see (d) of FIG. 6). An Au film having a film thickness of 40 nm was formed on the organic semiconductor layer 6 by use of a vacuum evaporation method in which a metallic mask (not illustrated) having a predetermined opening was used. A distance (channel length) between the source electrode 4 and the drain electrode 5 thus prepared was 50 μm, and a length (channel length) of each of the electrodes facing each other was 1000 μm.

### Example 5

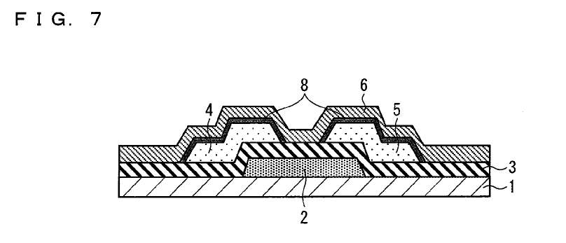

In Example 5, an organic thin-film transistor having the same arrangement as that of a field effect transistor illustrated in FIG. 7 was produced. FIG. 7 is a cross-sectional view illustrating a main part of a field effect transistor of another example, to which the present invention is applicable. As illustrated in FIG. 7, an organic thin-film transistor produced in the present example had an arrangement in which a gate electrode 2, a gate insulating film 3, a source electrode 4/drain electrode 5, a surface modification layer 8 (hydrophilic film), and an organic semiconductor layer 6 were provided on a substrate 1 in this order so that (i) the gate electrode 2 was covered with the gate insulating film 3, and (ii) the surface modification layer 8 was provided on a surface of the source electrode 4/drain electrode 5.

As the gate insulating film 3, a hydrophilic silicon oxide (SiO₂) film was used. With the arrangement, the gate insulating film 3 of the present example served as a hydrophilic film. Accordingly, the organic semiconductor layer 6 of the present example was formed on (i) the gate insulating film 3 serving as a hydrophilic film, and (ii) the surface modification layer 8 serving as a hydrophilic film.

The surface modification layer 8 was a film having a surface having a hydrophilic substituent group, and was formed by causing the source electrode 4 and the drain electrode 5 to be subjected to surface modification.

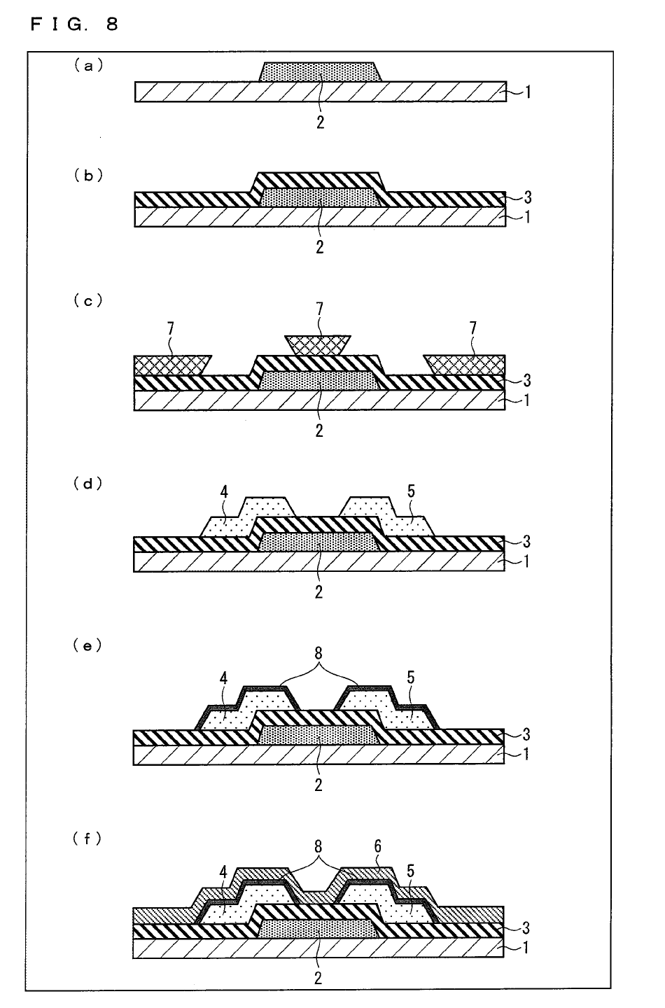

The following description deals with a method of producing an organic thin-film transistor in accordance with the present example, with reference to (a) through (f) of FIG. 8. Each of (a) through (f) of FIG. 8 is a cross-sectional view illustrating a step of a process of producing a field effect transistor of another example, to which the present invention is applicable.

First, the gate electrode 2 was provided on the substrate 1 (see (a) of FIG. 8). As the substrate 1, a glass substrate (Eagle 2000, film thickness: 0.5 mm, manufactured by Corning Incorporated) was used. Further, as the gate electrode 2, an AlSi alloy in which 10% of silicon was added to aluminum (Al) was used. By use of a sputtering method employing a target metal made from the AlSi alloy, a metallic film was formed on the substrate 1. The metallic film was made from the AlSi alloy and had a film thickness of 40 nm. Then, the metallic film was subjected to photolithography and etching, so as to be in a desired pattern. The gate electrode 2 illustrated in (a) of FIG. 8 was thus formed.

Next, the gate insulating film 3 was formed (see (b) of FIG. 8). As the gate insulating film 3, a silicon oxide film (SiO₂) was used. By use of the sputtering method, the silicon oxide film having a film thickness of 300 nm was formed on the gate electrode 2. The gate insulating film 3 was thus formed.

Next, in order to form the source electrode 4 and the drain electrode 5 by use of a liftoff technique, a photoresist film 7 having an opening section was formed (see (c) of FIG. 8). As the photoresist film 7, a negative photoresist (ZPN1150, manufactured by ZEON CORPORATION) for a liftoff process was used. By use of a spin coat method, a resist film having a thickness of 4 μm was formed on the gate insulating film 3. After that, the resist film was subjected to an exposure process and a development process by use of a photolithography method. The photoresist film 7 having a desired opening section was thus formed.

Next, the source electrode 4 and the drain electrode 5 were formed (see (d) of FIG. 8). A contact layer (not illustrated) made from chromium (Cr) and a metallic film made from gold (Au) were formed on the photoresist film 7 by use of a vacuum evaporation method. Specifically, a Cr film having a film thickness of 2 nm was formed as the contact layer, and then an Au film having a film thickness of 40 nm was formed successively. After the Au film was formed, a liftoff process was carried out so as to remove away (i) the photoresist film 7 provided on the gate insulating film 3 and (ii) an unnecessary part of the Au film/Cr film formed on the photoresist film 7. In the liftoff process, the substrate was immersed in an organic solvent such as acetone. A distance (channel length) between the source electrode 4 and the drain electrode 5 was 20 μm, and a length (channel width) of each of the electrodes facing each other was 1000 μm.

Next, a surface modification layer 8 was formed (see (e) of FIG. 8). The substrate on which the source electrode 4 and the drain electrode 5 were provided was immersed in an ethanol solution (10 mg/mL) of 2-aminoethanethiol for 5 hours. After that, the substrate was washed with isopropyl alcohol, and was dried in a dry nitrogen gas stream. The surface modification layer was thus formed. With the arrangement, both a surface of the source electrode 4 and a surface of the drain electrode 5 had a hydrophilic property.

Next, the organic semiconductor layer 6 was formed (see (f) of FIG. 8). As the organic semiconductor layer 6, a composition for an organic semiconductor layer, containing Compound 1 synthesized in Example 2, was used. As the composition for an organic semiconductor layer, a solution of Compound 1 (concentration: 0.5 wt %), in which toluene was used as a solvent, was used. The solution of the composition for an organic semiconductor layer was dropped, by use of a dispenser (not illustrated), on (i) the surface modification layer 8 provided on the source electrode 4 and the drain electrode 5, and (ii) the gate insulating film 3 sandwiched between the source electrode 4 and the drain electrode 5. Then, the solution was dried slowly in a gas of saturated chloroform by use of a casting method. The organic semiconductor layer 6 was thus formed. The organic semiconductor layer 6 had a thickness of approximately 40 nm.

### Example 6

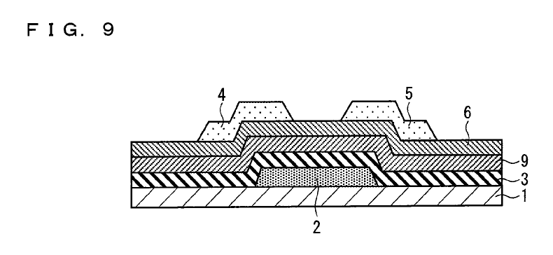

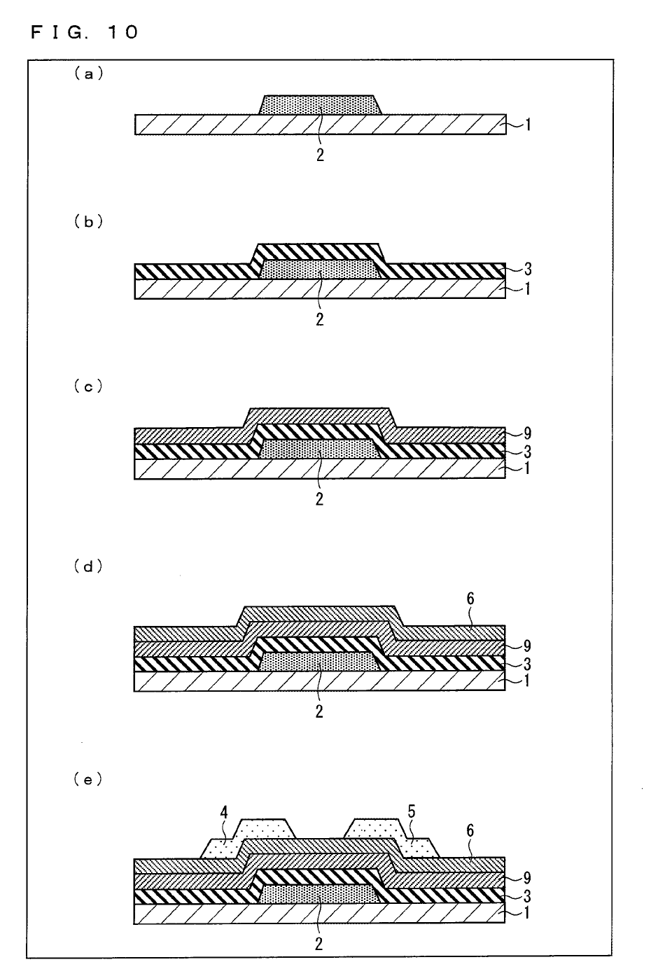

In Example 6, an organic thin-film transistor had the same arrangement as that of an organic thin-film transistor illustrated in FIG. 9 was produced. FIG. 9 is a cross-sectional view illustrating a main part of a field effect transistor of another example, to which the present invention is applicable. The organic thin-film transistor produced in the present example had an arrangement in which (i) a gate electrode 2, a gate insulating film 3, a hydrophilic polymer layer 9 (hydrophilic film), and an organic semiconductor layer 6 were provided on a substrate 1 in this order so that the gate electrode 2 was covered with the gate insulating film 3, and (ii) a source electrode 4 and a drain electrode 5 were provided on the organic semiconductor layer 6 (see FIG. 9).

In the present example, the organic semiconductor layer 6 was formed on the hydrophilic polymer layer 9 which (i) was provided on the gate insulating film 3 and (ii) served as a hydrophilic film.

The following description deals with a method of producing an organic thin-film transistor in accordance with the present example, with reference to (a) through (e) of FIG. 10. Each of (a) through (e) of FIG. 10 is a cross-sectional view illustrating a step of a process of producing a field effect transistor of another example, to which the present invention is applicable.

First, the gate electrode 2 was formed on the substrate 1 (see (a) of FIG. 10). As the substrate 1, a glass substrate (Eagle 2000, thickness: 0.5 mm, manufactured by Corning Incorporated) was used. Further, as the gate electrode 2, an AlSi alloy in which 10% of silicon was added to aluminum (Al) was used. By use of a sputtering method employing a target metal made from the AlSi alloy, a metallic film was formed on the substrate 1. The metallic film was made from the AlSi alloy and had a thickness of 40 nm. Then, the metallic film was subjected to photolithography and etching, so as to be in a desired shape. The gate electrode 2 illustrated in (a) of FIG. 10 was thus formed.

Next, the gate insulating film 3 was formed (see (b) of FIG. 10). As the gate insulating film 3, a silicon oxide film (SiO₂) was used. By use of the sputtering method, the silicon oxide film having a thickness of 300 nm was formed on the gate electrode 2. The gate insulating film 3 was thus formed.

Next, the hydrophilic polymer layer 9 was formed (see (c) of FIG. 10). By use of a spin coat method employing an aqueous solution of polyvinyl alcohol (concentration: 10 wt %) serving as a hydrophilic polymer, the hydrophilic polymer layer 9 was formed on the gate insulating film 3.

Next, the organic semiconductor layer 6 is formed (see (d) of FIG. 10). As the organic semiconductor layer 6, a composition for an organic semiconductor layer, containing Compound 1 synthesized in Example 2, was used. As the composition for an organic semiconductor layer, a solution of Compound 1 (concentration 0.5 wt %), in which toluene was used as a solvent, was used. By use of a spin coat method (the number of rotations: 1500 rpm), the organic semiconductor layer 6 was formed on the hydrophilic polymer layer 9. The organic semiconductor layer 6 thus formed had a thickness of approximately 40 nm.

Next, the source electrode 4 and the drain electrode 5 were formed (see (e) of FIG. 10). An Au film having a film thickness of 40 nm was formed on the organic semiconductor layer 6 via a metallic mask (not illustrated) having a predetermined opening section by use of a vacuum evaporation method. A distance (channel length) between the source electrode 4 and the drain electrode 5 was 50 μm, and a length (channel width) of each of the electrodes facing each other was 1000 μm.

The embodiments and concrete examples of implementation discussed in the foregoing detailed explanation serve solely to illustrate the technical details of the present invention, which should not be narrowly interpreted within the limits of such embodiments and concrete examples, but rather may be applied in many variations within the spirit of the present invention, provided such variations do not exceed the scope of the patent claims set forth below.

### INDUSTRIAL APPLICABILITY

A compound of the present invention is suitably used in a semiconductor device such as a field effect transistor and a solar cell.

### REFERENCE SIGNS LIST

2: Gate electrode
3: Gate insulating film
4: Source electrode
5: Drain electrode
6: Organic semiconductor layer
8: Surface modification layer (hydrophilic film)
9: Hydrophilic polymer layer (hydrophilic film)

1. A compound represented by the following formula (1): 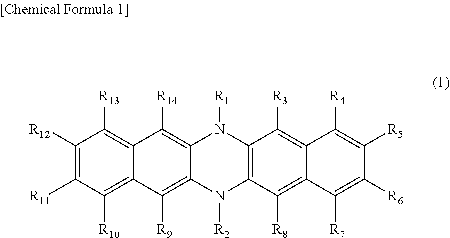
(where: R₁ and R₂ represent, independently, a substitutable C₁ to C₂₀ aliphatic hydrocarbon group; and R₃ through R₁₄ represent, independently, one of a hydrogen atom, a halogen atom, a substitutable C₁ to C₂₀ aliphatic hydrocarbon group, and a substitutable aromatic hydrocarbon group).

2. The compound as set forth in claim 1, wherein:
in the formula (1), R₁ and R₂ represent, independently, a substitutable C₆ to C₂₀ aliphatic hydrocarbon group, and R₃ through R₁₄ represent a hydrogen atom.

3. The compound as set forth in claim 2, wherein:
in the formula (1), R₁ and R₂ represent an n-hexyl group.

4. A field effect transistor comprising:
an organic semiconductor layer containing a compound recited in claim 1.

5. The field effect transistor as set forth in claim 4, wherein:
the organic semiconductor layer is provided on a hydrophilic film.

6. The field effect transistor as set forth in claim 4, wherein:
the field effect transistor includes a gate electrode, a gate insulating film, a source electrode, and a drain electrode, the gate electrode being covered with the gate insulating film;
the organic semiconductor layer is provided on the gate insulating film; and
the source electrode and the drain electrode are provided on the organic semiconductor layer so as to be in contact with the organic semiconductor layer.

7. The field effect transistor as set forth in claim 4, wherein:
the field effect transistor includes a gate electrode, a gate insulating film, a source electrode, and a drain electrode, the gate electrode being covered with the gate insulating film, the source electrode and the drain electrode being provided on the gate insulating film; and
the organic semiconductor layer is provided so as to cover the source electrode and the drain electrode.

8. The field effect transistor as set forth in claim 4, wherein:
the organic semiconductor layer is formed by application of the compound.

9. The field effect transistor as set forth in claim 4, wherein:
the organic semiconductor layer is formed by evaporation of the compound.

10. A method of producing a field effect transistor including an organic semiconductor layer containing a compound recited in claim 1, the method comprising the step of:
forming the organic semiconductor layer with the use of a composition containing the compound by use of one of a dipping method, a spin coat method, a casting method, an ink-jet method, and a print method,
the composition containing at least one selected from the group consisting of toluene, chlorobenzene, dichlorobenzene, trichlorobenzene, dichloromethane, and chloroform.

11. The method as set forth in claim 10, wherein:
the composition contains toluene.

12. A solar cell comprising:
a p-type semiconductor layer containing a compound recited in claim 1.

13. The solar cell as set forth in claim 12, wherein:
the p-type semiconductor layer is provided on a hydrophilic film.

14. The solar cell as set forth in claim 12, wherein:
the p-type semiconductor layer is formed by application of the compound.

15. The solar cell as set forth in claim 12, wherein:
the p-type semiconductor layer is formed by evaporation of the compound.

16. A method of producing a solar cell including a p-type semiconductor layer containing a compound recited in claim 1, the method comprising the step of:
forming the p-type semiconductor layer with the use of a composition containing the compound by use of one of a dipping method, a spin coat method, a casting method, an ink-jet method, and a print method,
the composition including at least one selected from the group consisting of toluene, chlorobenzene, dichlorobenzene, trichlorobenzene, dichloromethane, and chloroform.

17. The method as set forth in claim 16, wherein:
the composition contains toluene.

18. A solar cell comprising:
an organic semiconductor layer containing a p-type semiconductor material and an n-type semiconductor material,
the p-type semiconductor material containing a compound recited in claim 1.

19. A composition for an organic semiconductor layer of a field effect transistor, comprising:
a compound recited in claim 1.

20. A composition for a p-type semiconductor layer of a solar cell, comprising:
a compound recited in claim 1.

21. A composition for an organic semiconductor layer of a solar cell, comprising:
a p-type semiconductor material; and
an n-type semiconductor material,
the p-type semiconductor material containing a compound recited in claim 1.
